(19) United States
(12) Patent Application Publication
(10) Pub. No.: US 2024/0410017 A1
(43) Pub. Date: Dec. 12, 2024

(54) ASSESSING AND TREATING MULTIPLE MYELOMA

(71) Applicant: Mayo Foundation for Medical Education and Research, Rochester, MN (US)

(72) Inventors: Surendra Dasari, Rochester, MN (US); Mithun V. Shah, Rochester, MN (US); Dong Chen, Rochester, MN (US)

(21) Appl. No.: 18/699,836

(22) PCT Filed: Nov. 14, 2022
(86) PCT No.: PCT/US2022/049813

§ 371 Date: Apr. 9, 2024

Related U.S. Application Data
(60) Provisional application No. 63/279,430, filed on Nov. 15, 2021.

(51) Int. Cl. C12Q 1/6886; A61K 31/198; A61K 31/255; A61K 31/4045; A61K 31/4184; A61K 31/454; A61K 31/475; A61K 31/497; A61K 31/635; A61K 31/675; A61K 31/69; A61K 31/704; A61K 31/7048; A61K 33/243; A61K 38/07; A61K 39/395; A61N 5/10; A61P 35/00; C12Q 1/6874

(52) U.S. Cl. CPC C12Q 1/6886; A61K 31/198; A61K 31/255; A61K 31/4045; A61K 31/4184; A61K 31/454; A61K 31/475; A61K 31/497; A61K 31/635; A61K 31/675; A61K 31/69; A61K 31/704; A61K 31/7048; A61K 33/243; A61K 38/07; A61K 39/3955; A61N 5/10; A61P 35/00; C12Q 1/6874; C12Q 2600/154

(57) ABSTRACT

This document relates to methods and materials for assessing and/or treating mammals (e.g., humans) having multiple myeloma (MM). For example, methods and materials that can be used to determine whether or not a mammal (e.g., a human) having MM is likely to develop one or more therapy-related myeloid neoplasms (t-MNs) are provided. This document also relates to methods and materials for treating a mammal (e.g., a human) having MM where the treatment is selected based, at least in part, on whether or not the mammal is likely to develop one or more t-MNs.

### CROSS-REFERENCE TO RELATED APPLICATIONS

This application claims the benefit of U.S. Patent Application Ser. No. 63/279,430, filed on Nov. 15, 2021. The disclosure of the prior application is considered part of (and is incorporated by reference in) the disclosure of this application.

### TECHNICAL FIELD

This document relates to methods and materials for assessing and/or treating mammals (e.g., humans) having multiple myeloma (MM). For example, methods and materials provided herein can be used to determine whether or not a mammal (e.g., a human) having MM is likely to develop one or more therapy-related myeloid neoplasms (t-MNs). This document also provides methods and materials for treating a mammal (e.g., a human) having MM where the treatment is selected based, at least in part, on whether or not the mammal is likely to develop one or more t-MNs.

### BACKGROUND INFORMATION

MM is the most common indication for stem cell transplant (SCT), with >9000 SCTs performed in the U.S. annually (D'Souza et al., “Current Uses and Outcomes of Hematopoietic Cell Transplantation (HCT),” CIBMTR Summary Slides (2017)). Of MM patients that receive SCT, approximately 2-7% patients develop a t-MN after a median of 5-6 years (Palumbo et al., The Lancet Oncology, 15 (3): 333-342 (2014)). MM patients, even those not treated with DNA-damaging therapies such as chemotherapy, radiation, and SCT, are at a 12.5-fold higher risk of developing t-MN compared to the general population (Mailankody et al., Blood, 118 (15): 4086-4092 (2011)). t-MN is one of the most lethal and aggressive malignancies with a survival of 6-12 months and no effective therapies (McNerney et al., Nat. Rev. Cancer, 17 (9): 513-527 (2017)).

### SUMMARY

This document provides methods and materials for assessing and/or treating MM. In some cases, this document provides methods and materials for determining whether or not a mammal (e.g., a human) having MM is likely to develop one or more t-MNs (e.g., in response to a particular cancer treatment such as a DNA-damaging cancer treatment). For example, a sample (e.g., a sample containing genomic DNA such as a bone marrow (BM) sample) obtained from a mammal having MM can be assessed to determine if the mammal is likely to develop one or more t-MNs in response to a particular cancer treatment (e.g., a DNA-damaging cancer treatment) based, at least in part, on the presence or absence of altered methylation (e.g., hypermethylation or hypomethylation) of one or more (e.g., one, two, three, four, five, or more) nucleic acid sequences in the sample. This document also provides methods and materials for treating a mammal (e.g., a human) having MM where the treatment is selected based, at least in part, on whether or not the mammal is likely to develop one or more t-MNs. For example, a mammal (e.g., a human) having MM can be administered one or more cancer treatments that are selected based, at least in part, on the presence or absence of altered methylation (e.g., hypermethylation or hypomethylation) of one or more (e.g., one, two, three, four, five, or more) nucleic acid sequences in the sample.

As demonstrated herein, the presence of altered methylation of a SSU72 homolog, RNA polymerase II CTD phosphatase (SSU72) nucleic acid, a ribosomal protein S6 kinase C1 (RPS6KC1) nucleic acid, a disks large homolog 2 (I) I. (2) nucleic acid, a otogelin like (OTOGL) nucleic acid, and/or a PR/SET domain 15 (PRMD15) nucleic acid in genomic DNA of a mammal having MM can be used to identify that mammal as being likely to develop one or more t-MNs (e.g., in response to a particular cancer treatment such as a DNA-damaging cancer treatment).

Having the ability to identify a mammal having MM as being likely to develop one or more t-MNs (e.g., in response to a particular cancer treatment such as a DNA-damaging cancer treatment) based, at least in part, on the presence or absence of altered methylation of a SSU72 nucleic acid, a RPS6KC1 nucleic acid, a DLG2 nucleic acid, an OTOGL nucleic acid, and/or a PRMD15 nucleic acid provides a unique and unrealized opportunity to provide an individualized approach in selecting cancer therapies that can minimize the risk of t-MN. For example, a mammal having MM and identified as being at high risk of developing one or more t-MNs as described herein can be selected for increased screening, counselling, and/or treatment using one of more cancer treatments that is/are not a DNA-damaging cancer treatment (e.g., treatment using one or more non-SCT modalities).

In general, one aspect of this document features methods for assessing a mammal having MM. The methods can include, or consist essentially of, (a) determining if a sample from a mammal having MM contains the presence or absence of (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, or (5) decreased methylation of a PRMI) 15 nucleic acid, (b) classifying the mammal as being likely to develop a t-MN in response to a DNA-damaging cancer treatment if the presence is determined; and (c) classifying the mammal as not being likely to develop the t-MN in response to the DNA-damaging cancer treatment if the absence is determined. The mammal can be a human. The sample can be a bone marrow sample or a blood sample. The presence or absence of increased methylation of the SSU72 nucleic acid can be determined. The presence or absence of increased methylation of the RPS6KC1 nucleic acid can be determined. The presence or absence of decreased methylation of the DLG2 nucleic acid can be determined. The presence or absence of increased methylation of the OTOGL nucleic acid can be determined. The presence or absence of decreased methylation of the PRMI) 15 nucleic acid can be determined. In some cases, the method can include determining the presence. For example, the method can include determining the presence of increased methylation of a SSU72 nucleic acid, the presence of increased methylation of a RPS6KC1 nucleic acid, the presence of decreased methylation of a DI. (2 nucleic acid, the presence of increased methylation of an OTOGL, nucleic acid, and the presence of decreased methylation of a PRMD15 nucleic acid, and can include classifying the mammal as being likely to develop said t-MN in response to the DNA-damaging cancer treatment. In some cases, the method can include determining the absence. For example, the method can include determining the absence of increased methylation of a SSU72 nucleic acid, the absence of increased methylation of a RPS6KC1 nucleic acid, the absence of decreased methylation of a DLG2 nucleic acid, the absence of increased methylation of an OTOGL nucleic acid, and the absence of decreased methylation of a PRMD15 nucleic acid, and can include classifying the mammal as not being likely to develop the t-MN in response to the DNA-damaging cancer treatment. The DNA-damaging cancer treatment can be radiation therapy. The DNA-damaging cancer treatment can be administration of a DNA-damaging chemotherapeutic agent. The DNA-damaging chemotherapeutic agent can be melphalan, cyclophosphamide, doxorubicin, busulfan, vincristine, VP-16, bendamustine, or cisplatin. The DNA-damaging cancer treatment can be a SCT (e.g., an autologous SCT).

In another aspect, this document features methods for treating a mammal having MM. The methods can include, or consist essentially of, (a) determining if a sample from a mammal having MM contains the presence of (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, or (5) decreased methylation of a PRMD15 nucleic acid; and (b) administering a cancer treatment to the mammal, where the cancer treatment is not a DNA-damaging cancer treatment. The mammal can be a human. The sample can be a bone marrow sample or a blood sample. The cancer treatment can be administration of a chemotherapeutic agent. The chemotherapeutic agent can be carfilzomib, pomalidomide, panobinostat, ixazomib, elotuzumab, daratumumab, isatuximab, selinexor, venetoclax, or belantamab mafodotin.

In another aspect, this document features methods for treating a mammal having MM. The methods can include, or consist essentially of, administering a cancer treatment to a mammal having MM and identified as having (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, or (5) decreased methylation of a PRMD15 nucleic acid in a sample obtained from the mammal, where the cancer treatment is not a DNA-damaging cancer treatment. The mammal can be a human. The sample can be a bone marrow sample or a blood sample. The cancer treatment can be administration of a chemotherapeutic agent. The chemotherapeutic agent can be carfilzomib, pomalidomide, panobinostat, ixazomib, elotuzumab, daratumumab, isatuximab, selinexor, venetoclax, or belantamab mafodotin.

In another aspect, this document features methods for treating a mammal having MM. The methods can include, or consist essentially of, (a) determining if a sample from a mammal having MM contains the absence of (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, and (5) decreased methylation of a PRMD15 nucleic acid; and (b) administering a DNA-damaging cancer treatment to the mammal. The mammal can be a human. The sample can be a bone marrow sample or a blood sample. The DNA-damaging cancer treatment can be radiation therapy. The DNA-damaging cancer treatment can be administration of a DNA-damaging chemotherapeutic agent. The DNA-damaging chemotherapeutic agent can be melphalan, cyclophosphamide, doxorubicin, busulfan, vincristine, VP-16, bendamustine, or cisplatin. The DNA-damaging cancer treatment can be a SCT (e.g., an autologous SCT).

In another aspect, this document features methods for treating a mammal having MN. The methods can include, or consist essentially of, administering a DNA-damaging cancer treatment to a mammal having MM and identified as lacking (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, and (5) decreased methylation of a PRMD15 nucleic acid in a sample obtained from the mammal. The mammal can be a human. The sample can be a bone marrow sample or a blood sample. The DNA-damaging cancer treatment can be radiation therapy. The DNA-damaging cancer treatment can be administration of a DNA-damaging chemotherapeutic agent. The DNA-damaging chemotherapeutic agent can be melphalan, cyclophosphamide, doxorubicin, busulfan, vincristine, VP-16, bendamustine, or cisplatin. The DNA-damaging cancer treatment can be a SCT (e.g., an autologous SCT).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1: Methylation differences in t-MN cases and controls at baseline and at follow-up. Histogram of number of CpGs vs. delta beta (methylation) were shown for different group comparisons. Basl stands for baseline. FolUp stands for Follow-up. Cont stands for control. t-MN cases showed hypomethylation at baseline when compared to control. This profile was distinctive among the four group comparisons.

FIG. 2: Machine learning model performance using baseline methylome data. Baseline methylation values were fed into a random forest-based machine learning method. Left panel shows the ROC curve for the model, and right panel shows the probabilistic score (t-MN case status) generated by the model for t-MN cases and controls.

### DETAILED DESCRIPTION

This document provides methods and materials that can be used to determine whether or not a mammal having MM is likely to develop one or more t-MNs (e.g., in response to a particular cancer treatment such as a DNA-damaging cancer treatment). For example, a sample (e.g., a sample containing genomic DNA) obtained from a mammal having MM can be assessed for the presence or absence of altered methylation (e.g., hypermethylation or hypomethylation) of one or more (e.g., one, two, three, four, five, or more) nucleic acid sequences in the sample to determine whether or not the mammal is likely to develop one or more t-MNs (e.g., in response to a particular cancer treatment such as a DNA-damaging cancer treatment). In some cases, the methods and materials provided herein also can include administering one or more cancer treatments (e.g., one or more cancer treatments selected based, at least in part, on whether or not the mammal is likely to develop one or more t-MNs in response to a particular cancer treatment such as a DNA-damaging cancer treatment) to a mammal having MM to treat the mammal

A mammal (e.g., a human) having MM can be assessed to determine whether or not the mammal is likely to develop one or more t-MNs (e.g., in response to a particular cancer treatment such as a DNA-damaging cancer treatment) by detecting the presence or absence of altered methylation (e.g., hypermethylation or hypomethylation) of one or more (e.g., one, two, three, four, five, or more) nucleic acid sequences in a sample (e.g., a sample containing genomic DNA) obtained from the mammal. As described herein, the presence of altered methylation of a SSU72 nucleic acid, a RPS6KC1 nucleic acid, a DLG2 nucleic acid, an OTOGL nucleic acid, and/or a PRMD15 nucleic acid in a sample obtained from a mammal having MM can be used to determine whether or not that mammal is likely to develop one or more t-MNs (e.g., in response to a particular cancer treatment such as a DNA-damaging cancer treatment).

Any appropriate mammal having MM can be assessed and/or treated as described herein. Examples of mammals that can have MM and can be assessed and/or treated as described herein include, without limitation, humans, non-human primates (e.g., monkeys), dogs, cats, horses, cows, pigs, sheep, mice, rats, rabbits, and goats.

When assessing and/or treating a mammal (e.g., a human) having MM as described herein, the MM can be any type of MM. A MM can be any stage of MM (e.g., stage I, stage II, or stage III). In some cases, a MM can be a relapsed/refractory multiple myeloma (RRMM).

In some cases, the methods described herein can include identifying a mammal (e.g., a human) as having MM. Any appropriate method can be used to identify a mammal as having MM. For example, blood tests (e.g., for the presence of M proteins, free light chains, and/or beta-2-microglobulins), urine tests (e.g., for the presence of M proteins), laboratory tests (e.g., fluorescence in situ hybridization (FISH)), imaging techniques (e.g., X-ray, magnetic resonance imaging (MRI), computerized tomography (CT) scanning, and positron emission tomography (PET) scanning), and biopsy techniques can be used to identify a mammal (e.g., a human) as having MM.

When assessing a mammal (e.g., a human) having MM for a likelihood of developing one or more t-MNs (e.g., in response to a particular cancer treatment such as a DNA-damaging cancer treatment) as described herein (based, at least in part, on the presence or absence of altered methylation of one or more nucleic acid sequences in a sample obtained from the mammal), the t-MN can be any type of t-MN. Examples of t-MNs include, without limitation, therapy-related acute myeloid leukemia (t-AML), therapy-related myelodysplastic syndrome (t-MDS), and therapy-related MDS/myeloproliferative neoplasms (t-MDS/MPN). In some cases, a t-MN can be as described elsewhere (see, e.g., Arber et al., Blood, 127 (20): 2391-2405 (2016)).

In some cases, a mammal (e.g., a human) having MM can be identified as likely to develop one or more t-MNs (e.g., in response to a particular cancer treatment such as a DNA-damaging cancer treatment) based, at least in part, on the presence of altered methylation of one or more (e.g., one, two, three, four, five, or more) nucleic acid sequences in a sample (e.g., a sample containing genomic DNA) obtained from the mammal. In some cases, altered methylation can be an increased level of methylation (e.g., hypermethylation) of one or more nucleic acid sequences in a sample (e.g., a sample containing genomic DNA) obtained from the mammal. The term “increased level” as used herein with respect to a level of methylation of a nucleic acid sequence in a sample refers to any level that is higher than a reference level of methylation of the nucleic acid sequence. In some cases, altered methylation can be a decreased level of methylation (e.g., hypomethylation) of one or more nucleic acid sequences in a sample (e.g., a sample containing genomic DNA) obtained from the mammal. The term “decreased level” as used herein with respect to a level of methylation of a nucleic acid sequence in a sample refers to any level that is lower than a reference level of methylation of the nucleic acid sequence. The term “reference level” as used herein with respect to a level of methylation of a nucleic acid sequence refers to the level of methylation of the nucleic acid sequence typically observed in a control sample. Control samples can include, without limitation, samples from one or more healthy mammals (e.g., healthy humans), one or more samples from a mammal (e.g., a human) prior to the mammal being diagnosed with MM, and one or more samples from a mammal (e.g., a human) having MM that is/are obtained prior to the mammal being treated for the MM (e.g., prior to undergoing SCT). It will be appreciated that levels of methylation from comparable samples are used when determining whether or not a particular level is an altered level of methylation.

A mammal (e.g., a human) having MM that is likely to develop one or more t-MNs (e.g., in response to a particular cancer treatment such as a DNA-damaging cancer treatment) can have altered methylation of any appropriate nucleic acid sequence(s). Examples of nucleic acid sequences that can have altered methylation and can be used to identify a mammal (e.g., a human) having MM as being likely to develop one or more t-MNs (e.g., in response to a particular cancer treatment such as a DNA-damaging cancer treatment) as described herein include, without limitation, SSU72, RPS6KC1, DLG2, OTOGL, and PRMD15 nucleic acid sequences. In some cases, a mammal (e.g., a human) having MM that is likely to develop one or more t-MNs (e.g., in response to a particular cancer treatment such as a DNA-damaging cancer treatment) can have altered methylation of one or more of the nucleic acid sequences set forth in Table 1.

In some cases, an altered level of methylation can be an increased level of methylation (e.g., hypermethylation) present on a SSU72 nucleic acid. For example, a SSU72 nucleic acid having an increased level of methylation can have at least 20% (e.g., about 20%, about 30%, about 40%, about 50%, about 60%, about 70%, about 80%, about 90%, about 95%, or more) higher methylation than a reference level of methylation typically observed on a SSU72 nucleic acid. In some cases, a SSU72 nucleic acid having an increased level of methylation can have a level of SSU72 nucleic acid methylation that is from about 25% to about 35% higher than a reference level of methylation typically observed on a SSU72 nucleic acid. In some cases, a reference level of methylation typically observed for a SSU72 nucleic acid can be that level observed in a population of at least 25 mammals (e.g., a random sampling of 25, 50, 100, or more humans). In some case, a reference level of methylation typically observed for a SSU72 nucleic acid in humans can be from about 60% methylation to about 65% methylation (e.g., from about 60% to about 65% of a SSU72 nucleic acid in a human is methylated).

In some cases, an altered level of methylation can be an increased level of methylation (e.g., hypermethylation) present on a RPS6KC1 nucleic acid. For example, a RPS6KC1 nucleic acid having an increased level of methylation can have at least 20% (e.g., about 20%, about 30%, about 40%, about 50%, about 60%, about 70%, about 80%, about 90%, about 95%, or more) higher methylation than a reference level of methylation typically observed on a RPS6KC1 nucleic acid. In some cases, a RPS6KC1 nucleic acid having an increased level of methylation can have a level of RPS6KC1 nucleic acid methylation that is from about 45% to about 55% higher than a reference level of methylation typically observed on a RPS6KC1 nucleic acid. In some cases, a reference level of methylation typically observed for a RPS6KC1 nucleic acid can be that level observed in a population of at least 25 mammals (e.g., a random sampling of 25, 50, 100, or more humans). In some case, a reference level of methylation typically observed for a RPS6KC1 nucleic acid in humans can be from about 15% methylation to about 20% methylation (e.g., from about 15% to about 20% of a RPS6KC1 nucleic acid in a human is methylated).

In some cases, an altered level of methylation can be a decreased level of methylation (e.g., hypomethylation) present on a DLG2 nucleic acid. For example, a DLG2 nucleic acid having a decreased level of methylation can have at least 20% (e.g., about 20%, about 30%, about 40%, about 50%, about 60%, about 70%, about 80%, about 90%, about 95%, or more) lower methylation than a reference level of methylation typically observed on a DLG2 nucleic acid. In some cases, a DLG2 nucleic acid having a decreased level of methylation can have a level of DLG2 nucleic acid methylation that is from about 25% to about 35% lower than a reference level of methylation typically observed on a DLG2 nucleic acid. In some cases, a reference level of methylation typically observed for a DLG2 nucleic acid can be that level observed in a population of at least 25 mammals (e.g., a random sampling of 25, 50, 100, or more humans). In some case, a reference level of methylation typically observed for a DLG2 nucleic acid in humans can be from about 80% methylation to about 85% methylation (e.g., from about 80% to about 85% of a DLG2 nucleic acid in a human is methylated).

In some cases, an altered level of methylation can be an increased level of methylation (e.g., hypermethylation) present on an OTOGL nucleic acid. For example, an OTOGL nucleic acid having an increased level of methylation can have at least 20% (e.g., about 20%, about 30%, about 40%, about 50%, about 60%, about 70%, about 80%, about 90%, about 95%, or more) higher methylation than a reference level of methylation typically observed on an OTOGL nucleic acid. In some cases, an OTOGL nucleic acid having an increased level of methylation can have a level of OTOGL nucleic acid methylation that is from about 35% to about 45% higher than a reference level of methylation typically observed on an OTOGL nucleic acid. In some cases, a reference level of methylation typically observed for an OTOGL nucleic acid can be that level observed in a population of at least mammals (e.g., a random sampling of 25, 50, 100, or more humans). In some case, a reference level of methylation typically observed for an OTOGL nucleic acid in humans can be from about 5% methylation to about 10% methylation (e.g., from about 5% to about 10% of a OTOGL nucleic acid in a human is methylated).

In some cases, an altered level of methylation can be a decreased level of methylation (e.g., hypomethylation) present on a PRMD15 nucleic acid. For example, a PRMD15 nucleic acid having a decreased level of methylation can have at least 20% (e.g., about 20%, about 30%, about 40%, about 50%, about 60%, about 70%, about 80%, about 90%, about 95%, or more) lower methylation than a reference level of methylation typically observed on a PRMD15 nucleic acid. In some cases, a PRMD15 nucleic acid having a decreased level of methylation can have a level of PRMD15 nucleic acid methylation that is from about 25% to about 35% lower than a reference level of methylation typically observed on a PRMD15 nucleic acid. In some cases, a reference level of methylation typically observed for a PRMD15 nucleic acid can be that level observed in a population of at least 25 mammals (e.g., a random sampling of 25, 50, 100, or more humans). In some case, a reference level of methylation typically observed for a PRMD15 nucleic acid in humans can be from about 55% methylation to about 60% methylation (e.g., from about 55% to about 60% of a PRMD15 nucleic acid in a human is methylated).

Any appropriate method can be used to identify the presence or absence of altered methylation of one or more nucleic acid sequences (e.g., SSU72, RPS6KC1, DLG2, OTOGL, and PRMD15 nucleic acid sequences). For example, methylation-sensitive high resolution melting (MS-HRM), methylation specific qPCR, bisulfite sequencing (e.g., reduced representation bisulfite sequencing (RRBS), high throughput methylation arrays, low-density methylation arrays, and whole genome bisulfite sequencing (WGBS)) can be used to identify the presence, absence, or level of methylation of a nucleic acid sequence. In some cases, the presence or absence of altered methylation of one or more nucleic acid sequences (e.g., SSU72, RPS6KC1, DLG2, OTOGL, and PRMD15 nucleic acid sequences) can be identified as described in Example 1.

Any appropriate sample from a mammal (e.g., a human) having MM can be assessed as described herein (e.g., to determine whether or not the mammal is likely to develop one or more t-MNs in response to a particular cancer treatment such as a DNA-damaging cancer treatment based, at least in part, on the presence or absence of altered methylation of one or more nucleic acid sequences in a sample obtained from the mammal). In some cases, a sample can be a biological sample. In some cases, a sample can contain one or more cancer cells. In some cases, a sample can contain one or more biological molecules (e.g., nucleic acids such as DNA and RNA, polypeptides, carbohydrates, lipids, hormones, and/or metabolites). For example, a sample can contain genomic DNA. Examples of samples that can be assessed as described herein include, without limitation, fluid samples (e.g., whole blood, serum, plasma, urine, and saliva), tissue samples (e.g., bone marrow samples), cellular samples (e.g., buccal swabs), samples containing peripheral blood mononuclear cells (PBMCs), and samples containing peripheral blood stem cells (PBSCs). A sample can be a fresh sample or a fixed sample (e.g., a formaldehyde-fixed sample or a formalin-fixed sample). In some cases, one or more biological molecules can be isolated from a sample. For example, nucleic acid (e.g., genomic DNA) can be isolated from a sample and can be assessed as described herein.

In some cases, a mammal (e.g., a human) having MM and identified as not being likely to develop one or more t-MNs (e.g., in response to a particular cancer treatment such as a DNA-damaging cancer treatment) as described herein (e.g., based, at least in part, on the absence of altered methylation of one or more of the nucleic acid sequences) can be selected for decreased (e.g., less frequent) screening for one or more t-MNs. For example, a mammal having MM and identified as having the absence of altered methylation of one or more nucleic acid sequences (e.g., SSU72, RPS6KC1, DLG2, OTOGL, and PRMD15 nucleic acid sequences) in a sample (e.g., a sample containing genomic DNA) obtained from the mammal can be screened for one or more t-MNs no more than every 12 months. In some cases, a mammal having MM and identified as having the absence of altered methylation of one or more nucleic acid sequences (e.g., SSU72, RPS6KC1, DLG2, OTOGL, and PRMD15 nucleic acid sequences) in a sample (e.g., a sample containing genomic DNA) obtained from the mammal can be screened for one or more t-MNs every 6 months to 12 months.

In some cases, a mammal (e.g., a human) having MM and identified as being likely to develop one or more t-MNs (e.g., in response to a particular cancer treatment such as a DNA-damaging cancer treatment) as described herein (e.g., based, at least in part, on the presence of altered methylation of one or more of the nucleic acid sequences) can be selected for increased (e.g., more frequent) screening for one or more t-MNs. For example, a mammal having MM and identified as having the presence of altered methylation of one or more nucleic acid sequences (e.g., SSU72, RPS6KC1, DLG2, OTOGL, and PRMD15 nucleic acid sequences) in a sample (e.g., a sample containing genomic DNA) obtained from the mammal can be screened for one or more t-MNs at least every 3 months. In some cases, a mammal having MM and identified as having the presence of altered methylation of one or more nucleic acid sequences (e.g., SSU72, RPS6KC1, DLG2, OTOGL, and PRMD15 nucleic acid sequences) in a sample (e.g., a sample containing genomic DNA) obtained from the mammal can be screened for one or more t-MNs every 3 months to 6 months.

This document also provides methods for treating a mammal (e.g., a human) having MM. In some cases, a mammal (e.g., a human) having MM and assessed as described herein (e.g., to determine whether or not the mammal is likely to develop one or more t-MNs in response to a particular cancer treatment such as a DNA-damaging cancer treatment based, at least in part, on the presence or absence of altered methylation of one or more nucleic acid sequences in a sample obtained from the mammal) can be administered or instructed to self-administer one or more (e.g., one, two, three, four, five, or more) cancer treatments, where the one or more cancer treatments are effective to treat the cancer within the mammal (e.g., while minimizing the risk of the mammal developing one or more t-MNs). For example, a mammal having MM can be administered or instructed to self-administer one or more cancer treatments selected based, at least in part, on whether or not the mammal is likely to develop one or more t-MNs in response to a particular cancer treatment such as a DNA-damaging cancer treatment (e.g., based, at least in part, on the presence or absence of altered methylation of one or more nucleic acid sequences such as SSU72, RPS6KC1, DLG2, OTOGL, and PRMD15 nucleic acid sequences).

In general, a cancer treatment for MM can include any appropriate MM cancer treatment. In some cases, a cancer treatment can include administering one or more cancer drugs (e.g., chemotherapeutic agents, targeted cancer drugs, immunotherapy drugs, corticosteroids, and hormones) to a mammal in need thereof and/or subjecting a mammal in need thereof to one or more cancer therapies. Examples of cancer drugs that can be administered to a mammal having MM can include, without limitation, bortezomib, lenalidomide (e.g., REVLIMID®), thalidomide, melphalan (e.g., ALKERAN®) such as melphalan flufenamide hydrochloride (e.g., MELFLUFEN®), cyclophosphamide (e.g., CYTOXAN®), doxorubicin (e.g., ADRIAMYCIN®) such as pegylated liposomal doxorubicin (e.g., DOXIL®), busulfan (e.g., MYLERAN®), vincristine (e.g., ONCOVIN®), VP-16 (e.g., ETOPOSIDE®), bendamustine (e.g., TREANDA®), cisplatin (e.g., PLATINOL®), carfilzomib, pomalidomide, panobinostat, ixazomib, elotuzumab, daratumumab, isatuximab, selinexor, venetoclax, belantamab mafodotin, and combinations thereof. Examples of cancer therapies that can be performed on a mammal having MM to treat the mammal include, without limitation, stem cell transplants (e.g., bone marrow transplants), radiation therapy, adoptive cell therapies (e.g., T cell therapies such as chimeric-antigen receptor (CAR)T cell therapies and bispecific T cell engager (BiTE) therapies), and monoclonal antibody therapy (e.g., administration of belantamab).

When treating a mammal (e.g., a human) having MM and identified as not being likely to develop one or more t-MNs (e.g., in response to a particular cancer treatment such as a DNA-damaging cancer treatment) as described herein (e.g., based, at least in part, on the absence of altered methylation of one or more nucleic acid sequences such as SSU72, RPS6KC1, DLG2, OTOGL, and PRMD15 nucleic acid sequences), the mammal can be administered or instructed to self-administer one or more (e.g., one, two, three, four, five, or more) DNA-damaging cancer treatments. For example, a mammal having MM and identified as having the absence of altered methylation of one or more nucleic acid sequences (e.g., SSU72, RPS6KC1, DLG2, OTOGL, and PRMD1S nucleic acid sequences) in a sample (e.g., a sample containing genomic DNA) obtained from the mammal can be administered or instructed to self-administer one or more DNA-damaging cancer treatments. In some cases, a DNA-damaging cancer treatment can be a SCT (e.g., a SCT followed by administration of lenalidomide). For example, a DNA-damaging cancer treatment can be an autologous SCT. For example, a DNA-damaging cancer treatment can be an allogeneic SCT. In some cases, a DNA-damaging cancer treatment can be a radiation therapy. In some cases, a DNA-damaging cancer treatment can be administering a DNA-damaging chemotherapeutic agent. For example, a DNA-damaging chemotherapeutic agent can be an anthracycline. For example, a DNA-damaging chemotherapeutic agent can be a leukemogenic agent. Examples of DNA-damaging chemotherapeutic agents that can be used as described herein include, without limitation, melphalan (e.g., ALKERAN®) such as melphalan flufenamide hydrochloride (e.g., MELFLUFEN®), cyclophosphamide (e.g., CYTOXAN®), doxorubicin (e.g., ADRIAMYCIN®) such as pegylated liposomal doxorubicin (e.g., DOXIL®), busulfan (e.g., MYLERAN®), vincristine (e.g., ONCOVIN®), VP-16 (e.g., ETOPOSIDE®), bendamustine (e.g., TREANDA®), and cisplatin (e.g., PLATINOL®).

When treating a mammal (e.g., a human) having MM and identified as being likely to develop one or more t-MNs (e.g., in response to a particular cancer treatment such as a DNA-damaging cancer treatment) as described herein (e.g., based, at least in part, on the presence of altered methylation of one or more nucleic acid sequences such as SSU72, RPS6KC1, DLG2, OTOGL, and PRMD15 nucleic acid sequences), the mammal can be administered or instructed to self-administer one or more (e.g., one, two, three, four, five, or more) alternative cancer treatments (e.g., one or more cancer treatments that are not a DNA-damaging cancer treatment). For example, a mammal having MM and identified as having the presence of altered methylation of one or more nucleic acid sequences (e.g., SSU72, RPS6KC1, DLG2, OTOGL, and PRMD15 nucleic acid sequences) in a sample (e.g., a sample containing genomic DNA) obtained from the mammal can be administered or instructed to self-administer one or more alternative cancer treatments that are not DNA-damaging cancer treatments. In some cases, an alternative cancer treatment that is not a DNA-damaging cancer treatment can be a T cell therapy (e.g., a CART cell therapy or BiTE therapies). In some cases, an alternative cancer treatment that is not a DNA-damaging cancer treatment can be a monoclonal antibody therapy (e.g., administration of belantamab). In some cases, an alternative cancer treatment that is not a DNA-damaging cancer treatment can include administering one or more cancer drugs (e.g., chemotherapeutic agents, targeted cancer drugs, immunotherapy drugs, and hormones) other than a DNA-damaging cancer treatment. Examples of cancer drugs that are not a DNA-damaging cancer treatment and that can be administered to a mammal having MM and identified as being likely to develop one or more t-MNs (e.g., in response to a particular cancer treatment such as a DNA-damaging cancer treatment) can include, without limitation, carfilzomib, pomalidomide, panobinostat, ixazomib, elotuzumab, daratumumab, isatuximab, selinexor, venetoclax, belantamab mafodotin, and combinations thereof.

When treating a mammal (e.g., a human) having MM and identified as being likely to develop one or more t-MNs (e.g., in response to a particular cancer treatment such as a DNA-damaging cancer treatment) as described herein (e.g., based, at least in part, on the presence of altered methylation of one or more nucleic acid sequences such as SSU72, RPS6KC1, DLG2, OTOGL, and PRMD15 nucleic acid sequences), the mammal is not subjected to any SCT (e.g., an autologous SCT or an allogeneic SCT). For example, a mammal having MM and identified as having the presence of altered methylation of one or more nucleic acid sequences (e.g., SSU72, RPS6KC1, DLG2, OTOGL, and PRMD15 nucleic acid sequences) in a sample (e.g., a sample containing genomic DNA) obtained from the mammal is not subjected to any SCT (e.g., a SCT alone or a SCT followed by administration of lenalidomide).

In some cases, when treating a mammal (e.g., a human) having MM as described herein, the treatment can be effective to treat the MM. For example, the number of cancer cells present within a mammal can be reduced using the methods and materials described herein. In some cases, the methods and materials described herein can be used to reduce the number of cancer cells present within a mammal having MM by, for example, 10, 20, 30, 40, 50, 60, 70, 80, 90, 95, or more percent. In some cases, the number of cancer cells present within a mammal does not increase. For example, the size (e.g., volume) of one or more tumors present within a mammal can be reduced using the methods and materials described herein. In some cases, the methods and materials described herein can be used to reduce the size of one or more tumors present within a mammal having MM by, for example, 10, 20, 30, 40, 50, 60, 70, 80, 90, 95, or more percent. In some cases, the size (e.g., volume) of one or more tumors present within a mammal does not increase.

In some cases, when treating a mammal (e.g., a human) having MM as described herein, the treatment can be effective to treat the MM while minimizing the risk of the human developing a t-MN. For example, the number of cancer cells present within a mammal can be reduced using the methods and materials described herein without the mammal developing a t-MN. In some cases, the methods and materials described herein can be used to reduce the number of cancer cells present within a mammal having MM by, for example, 10, 20, 30, 40, 50, 60, 70, 80, 90, 95, or more percent without the mammal developing a t-MN. In some cases, the number of cancer cells present within a mammal does not increase. For example, the size (e.g., volume) of one or more tumors present within a mammal can be reduced using the methods and materials described herein. In some cases, the methods and materials described herein can be used to reduce the size of one or more tumors present within a mammal having MM by, for example, 10, 20, 30, 40, 50, 60, 70, 80, 90, 95, or more percent without the mammal developing a t-MN. In some cases, the size (e.g., volume) of one or more tumors present within a mammal does not increase.

In some cases, when treating a mammal (e.g., a human) having MM as described herein, the treatment can be effective to improve survival of the mammal. For example, the methods and materials described herein can be used to improve disease-free survival (e.g., relapse-free survival). For example, the methods and materials described herein can be used to improve progression-free survival. For example, the methods and materials described herein can be used to improve the survival of a mammal having MM by, for example, 10, 20, 30, 40, 50, 60, 70, 80, 90, 95, or more percent. For example, the methods and materials described herein can be used to improve the survival of a mammal having MM by, for example, at least 6 months (e.g., about 6 months, about 8 months, about 10 months, about 1 year, about 1.5 years, about 2 years, about 2.5 years, or about 3 years).

In some cases, when treating a mammal (e.g., a human) having MM as described herein, the treatment can be effective to improve survival of the mammal while minimizing the risk of the human developing a t-MN. For example, the methods and materials described herein can be used to improve disease-free survival (e.g., relapse-free survival) without the mammal developing a t-MN. For example, the methods and materials described herein can be used to improve progression-free survival without the mammal developing a t-MN. For example, the methods and materials described herein can be used to improve the survival of a mammal having MM by, for example, 10, 20, 30, 40, 50, 60, 70, 80, 90, 95, or more percent without the mammal developing a t-MN. For example, the methods and materials described herein can be used to improve the survival of a mammal having MM by, for example, at least 6 months (e.g., about 6 months, about 8 months, about 10 months, about 1 year, about 1.5 years, about 2 years, about 2.5 years, or about 3 years) without the mammal developing a t-MN.

In some cases, when treating a mammal (e.g., a human) having MM as described herein, the treatment can be effective to reduce or eliminate one or more symptoms of the MM. Examples of symptoms of MM include, without limitation, bone pain, nausea, constipation, loss of appetite, mental fogginess or confusion, fatigue, frequent infections, weight loss, weakness or numbness in your legs, excessive thirst, pain at the site of myeloma, inadequate urination, and changes in mental status. For example, the methods and materials described herein can be used to reduce one or more symptoms within a mammal having MM by, for example, 10, 20, 30, 40, 50, 60, 70, 80, 90, 95, or more percent.

In some cases, when treating a mammal (e.g., a human) having MM as described herein, the treatment can be effective to reduce or eliminate one or more symptoms of the MM while minimizing the risk of the human developing a t-MN. Examples of symptoms of MM include, without limitation, bone pain, nausea, constipation, loss of appetite, mental fogginess or confusion, fatigue, frequent infections, weight loss, weakness or numbness in your legs, excessive thirst, pain at the site of myeloma, inadequate urination, and changes in mental status. For example, the methods and materials described herein can be used to reduce one or more symptoms within a mammal having MM by, for example, 10, 20, 30, 40, 50, 60, 70, 80, 90, 95, or more percent without the mammal developing a t-MN.

In some cases, when treating a mammal (e.g., a human) having MM as described herein, the treatment can be effective to reduce or eliminate one or more complications associated with the MM. Examples of complications associated with MM include, without limitation, bone pain, kidney complications, infections, bone loss, anemia, encephalopathy, changes in mental status, dehydration, damage to kidneys (e.g., temporary or permanent damage to kidneys), inadequate heart function, and autonomic neuropathy. For example, the methods and materials described herein can be used to reduce one or more complications associated with MM within a mammal having MM by, for example, 10, 20, 30, 40, 50, 60, 70, 80, 90, 95, or more percent.

In some cases, when treating a mammal (e.g., a human) having MM as described herein, the treatment can be effective to reduce or eliminate one or more complications associated with the MM while minimizing the risk of the human developing a t-MN. Examples of complications associated with MM include, without limitation, bone pain, kidney complications, infections, bone loss, anemia, encephalopathy, changes in mental status, dehydration, damage to kidneys (e.g., temporary or permanent damage to kidneys), inadequate heart function, and autonomic neuropathy. For example, the methods and materials described herein can be used to reduce one or more complications associated with MM within a mammal having MM by, for example, 10, 20, 30, 40, 50, 60, 70, 80, 90, 95, or more percent without the mammal developing a t-MN.

In some cases, a mammal (e.g., a human) having MM can be monitored for the development of a t-MN. Any appropriate method can be used to determine whether or not a mammal (e.g., a human) has developed t-MN. For example, clinical monitoring of patient medical history, physical examination, blood tests, lactate dehydrogenase levels, peripheral blood smear exam, peripheral blood flow cytometry, peripheral blood genetic testing, bone marrow biopsy morphology exam, bone marrow flow cytometry, bone marrow genetic testing, biopsy of a mass for morphology exam, biopsy of a mass for flow cytometry, biopsy of a mass for next-generation sequencing, biopsy of a mass for fluorescence in situ hybridization, and/or biopsy of a mass for chromosome analysis can be used to determine whether or not a mammal (e.g., a human) has developed t-MN.

In some cases, a course of treatment, the number of cancer cells present within a mammal, and/or the severity of one or more symptoms related to the condition being treated (e.g., cancer) can be monitored. Any appropriate method can be used to determine whether or not the number of cancer cells present within a mammal is reduced. For example, imaging techniques can be used to assess the number of cancer cells present within a mammal.

The invention will be further described in the following examples, which do not limit the scope of the invention described in the claims.

### EXAMPLES

### Example 1: Methylation-Based Models for Risk Assessment of t-MNs

This Example describes a methylome analysis of MM patients and the identification of genes whose differentially methylated CpGs (DMC) can be used to predict possible future t-MN.

### Methods

Fifty two (52) MM patients who underwent autologous SCT and developed t-MN were identified. Paired bone marrow (BM) samples were obtained from patients that developed t-MN (referred to as t-MN cases) prior to undergoing SCT and at the time of t-MN diagnosis (n=13). Paired BM samples were also obtained from matched controls who underwent SCT but did not develop t-MN (n=29) despite a median follow up of 9 years (IQR 7.7-11). Pre-SCT samples are referred to as “baseline” (for both t-MN cases and controls) and follow up samples of controls are referred to as “follow-up.”

### Methylation Status Analysis

Methylation status of genomic DNA was assessed using reduced representation bisulfite sequencing (RRBS). Libraries were prepared using 100 ng of genomic DNA using the NuGen RRBS Ovation Kit (NuGen, Redwood City, CA). Briefly, dsDNA was digested with Msp1 and indexed methylated adaptors were ligated to the digested fragments with T4 DNA ligase. The ligated DNA was repaired with Final Repair mix. Bisulfite modification was performed using EZ-DNA Methylation Kit (Zymo Research, Irvine, CA). Bisulfite-modified product was amplified with PCR and purified. Completed libraries were pooled and to generate ˜40-100 million reads per sample. Base-calling was performed using Illumina's RTA version 2.7.3.

### Data Analysis and Modeling

CpG loci level methylation was computed for each sample. Methylation values of ˜100K loci were subjected to a supervised and machine learning-based unsupervised analysis. In supervised analysis, CpC loci that are differentially methylated between t-MN cases vs. controls (at baseline), t-MN cases vs. controls (follow up), t-MN cases (baseline vs. controls), and controls (baseline vs. follow up) were computed. A total of 22 loci that were differentially methylated between t-MN cases and controls both at baseline and at follow up were then fed into a multivariate model to distinguish t-MN cases from control using their baseline methylation status. In machine learning-based approach, baseline methylation status of all ˜100K loci observed t-MN cases and controls were fed into a variable selection method based on coefficient of variation of each loci. Loci with most variability (>50%) were then fed into a random forest to build a model that can distinguish between t-MN cases vs controls.

### Results

Supervised and unsupervised (machine learning-based) strategies were followed to test whether there are any methylation differences at baseline (i.e., before therapy) that can differentiate between t-MN cases vs. controls.

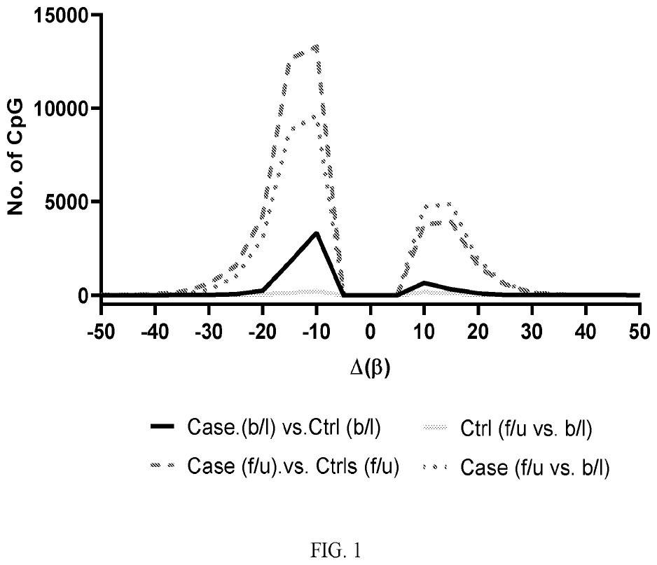

In a supervised strategy, four group comparisons were performed: cases vs. controls (at baseline), t-MN cases vs. controls (follow up), t-MN cases (baseline vs. controls), and controls (baseline vs. follow up). t-MN cases had a distinct methylation signature compared to controls (at both baseline and at follow up; FIG. 1). Twenty-two genes were identified that had differentially methylated CpGs (DMCs) between t-MN cases and controls at both at baseline and at follow up (Table 1), suggesting a role for those DMCs in t-MN pathogenesis. A literature review of the genes showed that none has a known role in myeloid pathogenesis. A stepwise discriminant multivariate analysis (JMP v14) using linear, common variance showed that methylation status of 5 CpGs accurately distinguished all t-MN cases from controls at baseline (Table 2). In t-MN cases, the methylation of three CpGs (associated with SSU72, RPS6KC1, and OTOGL) was elevated while the methylation of two CpGs (associated with DLG2 and PRDM15) was reduced relative to control samples (Table 3).

TABLE 1
DMCs between cases and controls that were identified both, at baseline and at follow up.

| | Associated | Difference in methylation | | | |
|---|---|---|---|---|---|
| Chromosome | gene | (%, case vs. control) | P-value | q-value | Gene ID |
| Chr1 | CAPZA1 | 43.76094903 | 3.03E−203 | 3.88E−198 | NM_006135 |
| Chr1 | NA | 26.84326844 | 6.13E−64 | 1.57E−59 | NA |
| Chr1 | SSU72 | 29.33496182 | 1.27E−97 | 8.91E−93 | NM_014188 |
| Chr1 | NA | −25.74047527 | 2.78E−59 | 6.30E−55 | NA |
| Chr1 | RPS6KC1 | 48.67636204 | 5.79E−172 | 5.57E−167 | NM_001136138 |
| Chr1 | NA | 37.06894699 | 3.31E−50 | 5.31E−46 | NA |
| Chr1 | ITGB3BP | 42.21237699 | 1.60E−216 | 3.09E−211 | NM_014288 |
| Chr2 | NA | 35.19366213 | 9.94E−120 | 8.50E−115 | NA |
| Chr5 | NA | 45.27500795 | 3.80E−103 | 2.92E−98 | NA |
| Chr5 | ATG10 | 39.72983067 | 3.33E−86 | 1.71E−81 | NM_001131028 |
| Chr8 | INTS9 | 28.48608245 | 4.99E−42 | 6.30E−38 | NM_001172562 |
| Chr8 | ARHGEF1 | 40.52212713 | 2.70E−67 | 7.68E−63 | NM_006421 |
| Chr9 | NA | −25.96406874 | 1.52E−22 | 3.47E−19 | NA |
| Chr11 | DLG2 | −31.71241845 | 3.15E−96 | 2.02E−91 | NM_001351274 |
| Chr12 | OTOGL | 39.96424314 | 4.65E−187 | 5.12E−182 | NM_001368062 |
| Chr16 | NA | −26.29333288 | 6.64E−55 | 1.25E−50 | NA |
| Chr19 | NA | 33.22433762 | 7.03E−61 | 1.69E−56 | NR_038237 |
| Chr20 | PYGB | 37.10649239 | 1.82E−29 | 8.76E−26 | NM_002862 |
| Chr20 | ABHD12 | −30.55291153 | 2.13E−78 | 8.62E−74 | NM_015600 |
| Chr20 | SLCO4A1 | −33.87485615 | 1.27E−29 | 6.14E−26 | NM_018354 |
| Chr21 | PRDM15 | −31.115714 | 8.55E−34 | 5.93E−30 | NR_104280 |
| Chr22 | APOL4 | −25.95827485 | 5.35E−28 | 2.35E−24 | NM_030643 |

TABLE 2
Discriminant multivariate analysis shows that the combination
of 5 DMC accurately predicts future t-MN.

Predicted phenotype

| Actual phenotype | Case | Control |
|---|---|---|
| Case | 13 | 0 |
| Control | 0 | 29 |

Case—patients that develop future t-MN;
control—age-, sex-, and MM-type matched controls

TABLE 3
List of most differentially methylated CpG loci between those
who developed t-MN (Cases) compared to matched controls.

| | | | Average % |
|---|---|---|---|
| | | Average % | methylation |
| | | methylation | in Controls |
| | Associated | in Cases | (reference) |
| CpG Locus | gene | (n = 13) | (n = 29 |
| chr1.1554290.1554290 | SSU72 | 93.80 | 63.46 |
| chr1.213151421.213151421 | RPS6KC1 | 57.17 | 17.36 |
| chr11.85484014.85484014 | DLG2 | 52.60 | 81.32 |
| chr12.80307513.80307513 | OTOGL | 43.90 | 9.90 |
| chr21.41823545.41823545 | PRDM15 | 29.71 | 57.83 |

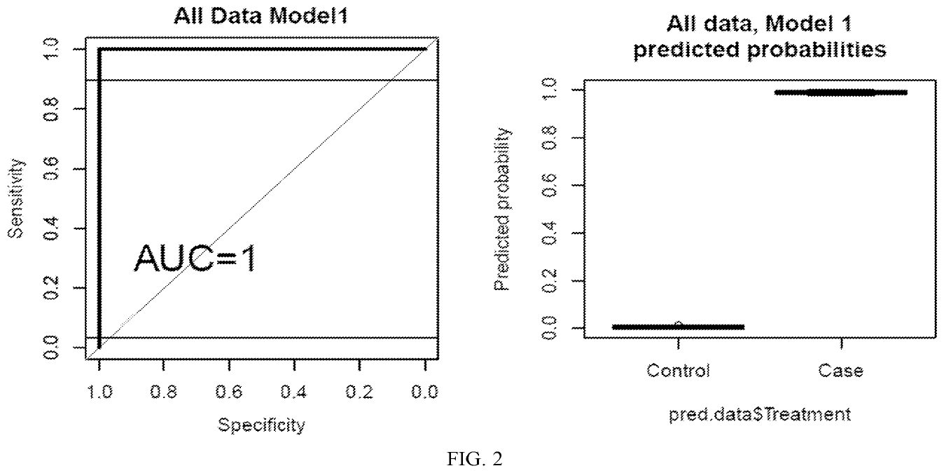

Independently, the baseline methylation status of all probes observed in both t-MN cases vs. controls was taken, and a machine learning method was utilized to choose probes to differentiate between the two groups. A random forest-based model was generated using the most informative probes and the performance of this model is shown in FIG. 2. It should be noted that the unsupervised method did not use follow up data for any modeling, which highlights the power of baseline methylation status alone in predicting t-MN.

Both the supervised and unsupervised (machine learning-based) analyses showed near perfect differentiation of t-MN cases vs. control at baseline when using bone marrow methylation status (Table 2 for supervised and FIG. 2 for machine learning model), and show that patients that develop t-MN have a strikingly different methylation signature even prior to undergoing SCT.

These results demonstrate that methylation status analysis can be used to accurately predict future t-MN, which allows for earlier risk stratification for t-MN and better management of these patients.

### Example 2: Treating MM

A biological sample (e.g., a BM sample or a blood sample) containing genomic DNA is obtained from a human having MM. The obtained sample is examined for the presence or absence of altered methylation of a SSU72 nucleic acid, a RPS6KC1 nucleic acid, a DLG2 nucleic acid, an OTOGL nucleic acid, and/or a PRMD15 nucleic acid.

If the presence of altered methylation of a SSU72 nucleic acid, a RPS6KC1 nucleic acid, a DLG2 nucleic acid, an OTOGL nucleic acid, and/or a PRMD15 nucleic acid is detected in the sample, then the human is identified as being likely to develop one or more t-MNs if administered a DNA-damaging cancer treatment, and the human is instead administered one or more alternative cancer treatments (e.g., one or more cancer treatments that are not a DNA-damaging cancer treatment).

The administered one or more alternative cancer treatments (e.g., one or more cancer treatments that are not a DNA-damaging cancer treatment) can reduce number of cancer cells within the human while minimizing the risk of the human developing a t-MN.

### Example 3: Treating MM

A biological sample (e.g., a BM sample or a blood sample) containing genomic DNA is obtained from a human having MM. The obtained sample is examined for the presence or absence of altered methylation of a SSU72 nucleic acid, a RPS6KC1 nucleic acid, a DLG2 nucleic acid, an OTOGL nucleic acid, and/or a PRMD15 nucleic acid.

If the absence of altered methylation of a SSU72 nucleic acid, a RPS6KC1 nucleic acid, a DLG2 nucleic acid, an OTOGL nucleic acid, and/or a PRMD15 nucleic acid is detected in the sample, then the human is identified as not being likely to develop one or more t-MNs if administered a DNA-damaging cancer treatment, and the human is administered one or more DNA-damaging cancer treatments.

The administered DNA-damaging cancer treatments can reduce number of cancer cells within the human while minimizing the risk of the human developing a t-MN.

### Example 4: Exemplary Embodiments

Embodiment 1. A method for assessing a mammal having multiple myeloma (MM), wherein said method comprises:
(a) determining if a sample from said mammal contains the presence of (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, or (5) decreased methylation of a PRMD15 nucleic acid, or the absence of (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, and (5) decreased methylation of a PRMD15 nucleic acid,
(b) classifying said mammal as being likely to develop a therapy-related myeloid neoplasm (t-MN) in response to a DNA-damaging cancer treatment if said presence is determined; and
(c) classifying said mammal as not being likely to develop said t-MN in response to said DNA-damaging cancer treatment if said absence is determined.

Embodiment 2. The method of embodiment 1, wherein said mammal is a human.

Embodiment 3. The method of any one of embodiments 1-2, wherein said sample is a bone marrow sample or a blood sample.

Embodiment 4. The method of any one of embodiments 1-3, wherein the presence or absence of increased methylation of said SSU72 nucleic acid is determined.

Embodiment 5. The method of any one of embodiments 1-3, wherein the presence or absence of increased methylation of said RPS6KC1 nucleic acid is determined.

Embodiment 6. The method of any one of embodiments 1-3, wherein the presence or absence of decreased methylation of said DLG2 nucleic acid is determined.

Embodiment 7. The method of any one of embodiments 1-3, wherein the presence or absence of increased methylation of said OTOGL nucleic acid is determined.

Embodiment 8. The method of any one of embodiments 1-3, wherein the presence or absence of decreased methylation of said PRMD15 nucleic acid is determined.

Embodiment 9. The method of any one of embodiments 1-8, wherein said method comprises determining said presence.

Embodiment 10. The method of any one of embodiments 1-9, wherein said method comprises determining the presence of increased methylation of a SSU72 nucleic acid, the presence of increased methylation of a RPS6KC1 nucleic acid, the presence of decreased methylation of a DLG2 nucleic acid, the presence of increased methylation of an OTOGL nucleic acid, and the presence of decreased methylation of a PRMD15 nucleic acid.

Embodiment 11. The method of embodiment 9 or 1 embodiment 0, wherein said method comprises classifying said mammal as being likely to develop said t-MN in response to said DNA-damaging cancer treatment.

Embodiment 12. The method of any one of embodiments 1-8, wherein said method comprises determining said absence.

Embodiment 13. The method of any one of embodiments 1-9, wherein said method comprises determining the absence of increased methylation of a SSU72 nucleic acid, the absence of increased methylation of a RPS6KC1 nucleic acid, the absence of decreased methylation of a DLG2 nucleic acid, the absence of increased methylation of an OTOGL nucleic acid, and the absence of decreased methylation of a PRMD15 nucleic acid.

Embodiment 14. The method of embodiment 12 or embodiment 13, wherein said method comprises classifying said mammal as not being likely to develop said t-MN in response to said DNA-damaging cancer treatment.

Embodiment 15. The method of any one of embodiments 1-14, wherein said DNA-damaging cancer treatment is radiation therapy.

Embodiment 16. The method of any one of embodiments 1-14, wherein said DNA-damaging cancer treatment is administration of a DNA-damaging chemotherapeutic agent.

Embodiment 17. The method of embodiment 16, wherein said DNA-damaging chemotherapeutic agent is selected from the group consisting of melphalan, cyclophosphamide, doxorubicin, busulfan, vincristine, VP-16, bendamustine, and cisplatin.

Embodiment 18. The method of any one of embodiments 1-14, wherein said DNA-damaging cancer treatment is a stem cell transplant (SCT).

Embodiment 19. The method of embodiment 18, wherein said SCT is an autologous SCT.

Embodiment 20. A method for treating a mammal having MM, wherein said method comprises:
(a) determining if a sample from said mammal contains the presence of (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, or (5) decreased methylation of a PRMD15 nucleic acid; and
(b) administering a cancer treatment to said mammal, wherein said cancer treatment is not a DNA-damaging cancer treatment.

Embodiment 21. A method for treating a mammal having MM, wherein said method comprises administering a cancer treatment to a mammal identified as having (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, or (5) decreased methylation of a PRMD15 nucleic acid in a sample obtained from said mammal, wherein said cancer treatment is not a DNA-damaging cancer treatment.

Embodiment 22. The method of any one of embodiments 20-21, wherein said mammal is a human.

Embodiment 23. The method of any one of embodiments 20-22, wherein said sample is a bone marrow sample or a blood sample.

Embodiment 24. The method of any one of embodiments 20-23, wherein said cancer treatment is administration of a chemotherapeutic agent.

Embodiment 25. The method of embodiment 24, wherein said chemotherapeutic agent is selected from the group consisting of carfilzomib, pomalidomide, panobinostat, ixazomib, elotuzumab, daratumumab, isatuximab, selinexor, venetoclax, and belantamab mafodotin.

Embodiment 26. A method for treating a mammal having MM, wherein said method comprises:
(a) determining if a sample from said mammal contains the absence of (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, and (5) decreased methylation of a PRMD15 nucleic acid; and
(b) administering a DNA-damaging cancer treatment to said mammal.

Embodiment 27. A method for treating a mammal having MM, wherein said method comprises administering a DNA-damaging cancer treatment to a mammal identified as lacking (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, and (5) decreased methylation of a PRMD15 nucleic acid in a sample obtained from said mammal.

Embodiment 28. The method of any one of embodiments 26-27, wherein said mammal is a human.

Embodiment 29. The method of any one of embodiments 26-28, wherein said sample is a bone marrow sample or a blood sample.

Embodiment 30. The method of any one of embodiments 26-29, wherein said DNA-damaging cancer treatment is radiation therapy.

Embodiment 31. The method of any one of embodiments 26-29, wherein said DNA-damaging cancer treatment is administration of a DNA-damaging chemotherapeutic agent.

Embodiment 32. The method of embodiment 31, wherein said DNA-damaging chemotherapeutic agent is selected from the group consisting of melphalan, cyclophosphamide, doxorubicin, busulfan, vincristine, VP-16, bendamustine, and cisplatin.

Embodiment 33. The method of any one of embodiments 26-29, wherein said DNA-damaging cancer treatment is a SCT.

Embodiment 34. The method of embodiment 33, wherein said SCT is an autologous SCT.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-19. (canceled)

20. A method for treating a mammal having MM, wherein said method comprises:
(a) determining if a sample from said mammal contains the presence of (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, or (5) decreased methylation of a PRMD15 nucleic acid; and
(b) administering a cancer treatment to said mammal, wherein said cancer treatment is not a DNA-damaging cancer treatment.

21. (canceled)

22. The method of claim 20, wherein said mammal is a human.

23. The method of claim 20, wherein said sample is a bone marrow sample or a blood sample.

24. The method of claim 20, wherein said cancer treatment is administration of a chemotherapeutic agent.

25. The method of claim 24, wherein said chemotherapeutic agent is selected from the group consisting of carfilzomib, pomalidomide, panobinostat, ixazomib, elotuzumab, daratumumab, isatuximab, selinexor, venetoclax, and belantamab mafodotin.

26. A method for treating a mammal having MM, wherein said method comprises:
(a) determining if a sample from said mammal contains the absence of (1) increased methylation of a SSU72 nucleic acid, (2) increased methylation of a RPS6KC1 nucleic acid, (3) decreased methylation of a DLG2 nucleic acid, (4) increased methylation of an OTOGL nucleic acid, and (5) decreased methylation of a PRMD15 nucleic acid; and
(b) administering a DNA-damaging cancer treatment to said mammal.

27. (canceled)

28. The method of claim 26, wherein said mammal is a human.

29. The method of claim 26, wherein said sample is a bone marrow sample or a blood sample.

30. The method of claim 26, wherein said DNA-damaging cancer treatment is radiation therapy.

31. The method of claim 26, wherein said DNA-damaging cancer treatment is administration of a DNA-damaging chemotherapeutic agent.

32. The method of claim 31, wherein said DNA-damaging chemotherapeutic agent is selected from the group consisting of melphalan, cyclophosphamide, doxorubicin, busulfan, vincristine, VP-16, bendamustine, and cisplatin.

33. The method of claim 26, wherein said DNA-damaging cancer treatment is a SCT.

34. The method of claim 33, wherein said SCT is an autologous SCT.
